(19) United States
(12) Patent Application Publication
(10) Pub. No.: US 2002/0161444 A1
(43) Pub. Date: Oct. 31, 2002

(54) Spine fusion implant

(76) Inventor: Gil-Woon Choi, Inchon-city (KR)

Correspondence Address: Diane Dunn McKay, Esq., Mathews, Collins, Shepherd & Gould, P.A., Suite 306, 100 Thanet Circle, Princeton, NJ, 08540, US

(21) Appl. No.: 10/127,631

(22) Filed: Apr. 22, 2002

(30) Foreign Application Priority Data
Apr. 28, 2001 (KR) 2001-023245

(51) Int. Cl. A61F 2/44

(52) U.S. Cl. 623/17.11

(57) ABSTRACT

The present invention provide a spine fusion implant comprising: a cylindrical main body having an extendable front end; a pressing body of a cap shape expanding the front end of the main body by receiving a pulling force; a closing body coupled to a rear end of the main body and serving as a guide member for a coupling of a fastening body; the fastening body coupled to the closing body and pulling the pressing body. In the present invention as mention above, the front end of the main body is extended by the pressing body and fastening body with the main body being inserted in a fused part during the operation so that the spine fusion implant is seated suitable for the front and rear space between adjacent spine bodied.

### BACKGROUND OF THE INVENTION

1. Field of the Invention

The present invention relates to a spine fusion implant used for a spine fusion operation, more specifically the spine fusion implant comprising a main body having an extendable front end, and a pressing body and fastening body enabling an extension of the front end, which minimizes a space occupied by the pressing body so as to be filled with a bony material and fusion facilitating agent as much more, and leads an effective dispersion of the bony material and fusion facilitating agent so that the spine fusion implant and a peripheral spine bodies are fused well, and a growth of the bone and the safety after an operation are increased.

2. Background of the Related Art

In the human body, the spine forms a curve of ‘Lasy-S’ by connecting a plurality of bodies of spine with a disk, one section of the spine consists of cylindrical body and the arch located backward, the posterior spinal arch comprises a spinal canal with a pair of pedicles, superior or inferior facets extending backwardly from the pedicles, laminas and a spinous process, and a nerve is located through the spinal canal.

In such a spine, when the disks connecting the bodies is degenerated and ruptured so that it is separated from regular position or a joint consist of a rear projection of the spine is damaged and degenerated, and when the external force is loaded to or press the nerve passing through the spinal canal with various reasons such that the spine has a deformity and displacement from the normal position, it is known that pains are caused.

In case of a patient of which a part of the spine, especially the disk is broken or damaged, an operation which fuses a spine by using an implant substituting for the broken or damaged disk, is required.

Generally, the spine fusion relating to the lordosis is performed by a bony bridging method using a space between adjacent spine bodies where the disk is located.

The bony bridging method is to form a bore penetrating the disk space between adjacent bodies, and then insert the implant into the bore so as to fuse the implant with adjacent bodies.

FIG. 7 is a perspective view showing a spine fusion implant of the prior art;

The spine fusion implant BI′ has a shape of band which includes through holes formed with a fixed distance along the circumference. And, holes 100 to which a driver D is coupled are formed at a front and rear end of the spine fusion implant BI′.

Especially, the front end of implant BI′ has a width wider than that of the rear end, so that the implant BI′ could be inserted and seated to be suitable for a front and rear spaces between adjacent bodies after implanting.

FIG. 8 and FIG. 9 are sectional view showing a inserting procedure of the prior art spine fusion implant.

Firstly, as shown in FIG. 8, the bore is formed in the disk space between the adjacent bodies P and P, the operation driver D is coupled into the hole 100 of the spine fusion implant BI′ , and then the spine fusion implant BI′ is inserted in the bore, being laid horizontally.

Since a height of the spine fusion implant BI′ is identical in the front and rear ends, the inserting proceeds easily.

Continuously, as shown in FIG. 9, the spine fusion implant BI′ is located at suitable position of portion to be fused and rotated by 90°, so that it could be seated appropriately in a wide front space and a relatively narrow rear space between the bodies P and P.

After the completion of the implant insertion such as the above, when a bony material or fusion facilitating agent is put in the spine fusion implant BI′ and fills between the adjacent bodies P and P, the spine fusion operation is finished.

This spine fusion implant BI′ is, as shown in FIG. 10, generally inserted both of the disk spaces between adjacent bodies P and P.

However, the spine fusion implant of prior art has many problems as follows.

First, the implant made to be suitable for the front and rear spaces between the adjacent bodies, is inserted laid horizontally and rotated by 90°. During the rotation of spine fusion implant, the spine bodies are cut and damaged unnecessarily by sharp edges of both side of the implant.

Second, after implanting operation, the space between the adjacent bodies is filled with the bony material or fusion facilitating agent. However, it is difficult to fill the space between adjacent bodies sufficiently before the operation due to the structural characteristics of the prior art implant. And it is not only difficult but also cumbersome and complex to fill the space through the hole of the implant after the operation. Therefore, the operation time is increased, and this could be a burden to the patient.

### SUMMARY OF THE INVENTION

The present invention is contemplated to solve the aforementioned problem, and it is an object of the present invention to provide a spine fusion implant which is seated suitable for a front and rear spaces between adjacent spine bodies by expanding the front end thereof after the completion of insertion, so that an improper damage of spine bodies is perfectly excluded, and uses a cylindrical main body allowing a sufficient amount of a bone before an operation so that the entire operation is performed safely and effectively.

It is an another object of the present invention to provide the spine fusion implant, which decreases a size of pressing body occupying a considerable space in the main body, maintaining the minimum function thereof so as to define an inner space to be filled with the bone widely as possible, and uses a screw type fastening member so as to disperse the bone filled therein toward peripheral spine bodies and increase a filling density of the bone, so that the spine fusion implant and the spine bodies are fused stably and rapidly, a bone growth and spine stability is increased, and a doctor could retain composure due to the easiness of operation.

To accomplish the above object, the present invention provide a spine fusion implant comprising: a cylindrical main body having an extendable front end; a pressing body of a cap shape expanding the front end of the main body by receiving a pulling force; a closing body coupled to a rear end of the main body and serving as a guide member for a coupling of a fastening body; the fastening body coupled to the closing body and pulling the pressing body.

In the present invention as mention above, the front end of the main body is extended by the pressing body and fastening body with the main body being inserted in a fused part during the operation so that the spine fusion implant is seated suitable for the front and rear space between adjacent spine bodied.

### BRIEF DESCRIPTION OF THE DRAWINGS

The features and advantages of the present invention will be more described specifically in the following description of preferred embodiments of the invention with reference to the accompanying drawings wherein:

FIG. 1 is a front view of a spine fusion implant according to the present invention;

FIG. 2 is an exploded sectional view of a spine fusion implant according to the present invention;

FIG. 3a-FIG. 3d are sectional view showing a inserting procedure of the spine fusion implant according to the present invention;

FIG. 4 is a perspective view showing another embodiment of the spine fusion implant according to the present invention;

FIG. 5 is an exploded perspective view showing another embodiment of the spine fusion implant according to the present invention;

FIG. 6 is a perspective view showing an expanded end portion of another embodiment of the spine fusion implant of FIG. 4 and FIG. 5;

FIG. 7 is a perspective view showing a spine fusion implant of the prior art;

FIG. 8 and FIG. 9 are sectional view showing a inserting procedure of the prior art spine fusion implant; and

FIG. 10 a rear view showing insertion position of the spine fusion implant.

### DETAILED DESCRIPTION OF THE PREFERRED EMBODIMENT

Reference will now be made in detail to the first and second embodiment of the present invention, examples of which are illustrated in the accompanying drawings. In explaining the present invention, the same names and reference numerals will be given to the same components, and explanations in the same will be omitted.

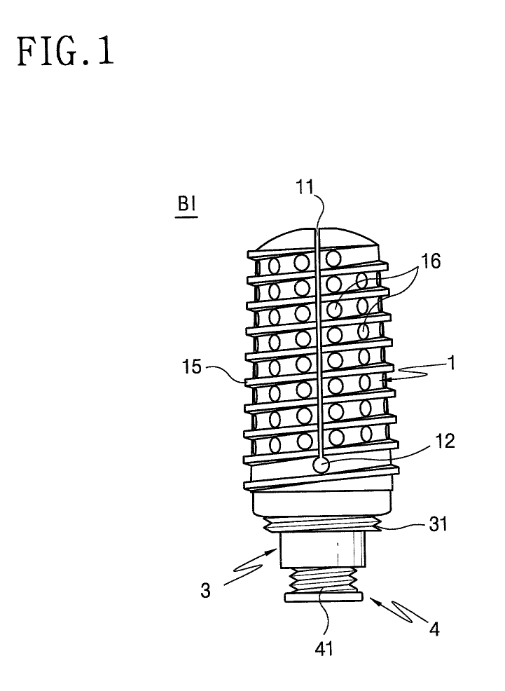

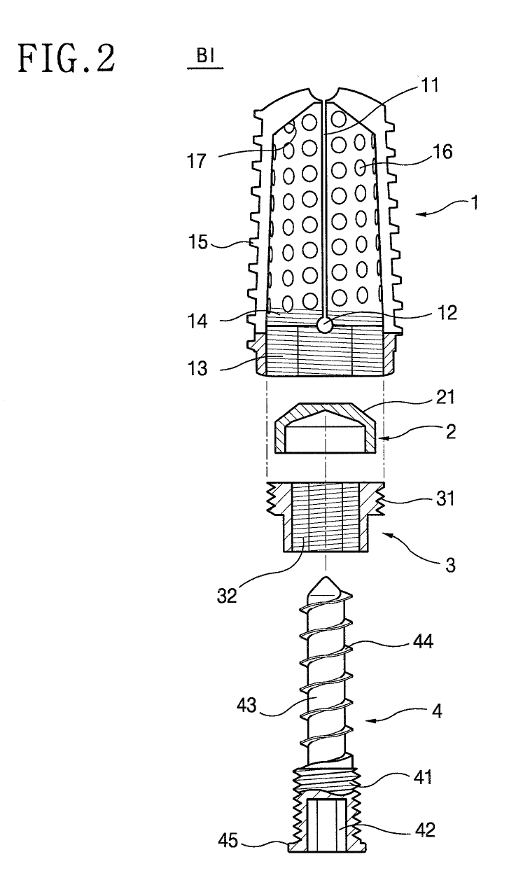

FIG. 1 illustrates a front view of a spine fusion implant according to the present invention, and FIG. 2 illustrates an exploded sectional view of the spine fusion implant according to the present invention.

A main body 1 comprises a cylinder having a spherical front end and an opened rear end, and an incision part 11 which extends from the front end to the rear end and is incised at least in one direction, is formed with the main body 1 so that the incision part 11 enables the expansion of front end.

This cylindrical structure of the main body 1 also makes it easy to fill a bone taken from the patient into the main body 1.

The incision part 11 is preferably a straight type or a cross type which separates the front end of the body into two of four pieces, and more number of the incision part 11 could be formed with the main body 1.

The strength decrease or breakage of main body 1 due to the expansion of front end is prevented by an incision maintaining hole 12 formed at an end of the incision part 11.

A driver mounting surface 13 having a shape of bolt heat is formed on an inner circumferential surface of the rear end in the main body 1 so as to be coupled with an operation driver, a threaded part 14 of a certain extent is formed at an inner side of the driver mounting surface 13, and a threaded part 31 of closing body 3 is coupled thereto.

Additionally, a spiral rib 15 is formed to projecting continuously on an outer circumferential surface of the main body 1 in length direction, and this rib 15 allows the easy insertion of the main body 1 and facilitates the growth of the bone by increased contacting surface with the bodies of spine.

Moreover, a plurality of holes 16 for bone fusion is provided to the entire surface of the main body 1, and this holes 16 serves to assist the internal growth of bone and the join of bone during the fusion procedure.

A reference numeral 17 indicates a compressed surface of ‘ˆ ’ shape which is formed at an inside of the front end so as to engage with a pressing body 2 described below.

Especially, the front-end diameter of main body 1 is smaller than the rear end diameter, for example by 1-2 mm to be inserted between the bodies of spine.

The pressing body 2 comprises a cap member, which has an inclined pressing surface 12 at a front end and is fixed temporarily by inserted on the compressed surface 17.

This pressing body 2 has a length to contact only with the compressed surface 17 of the main body 1, i.e. has a minimum volume occupying an inner space of the main body 1, so that the larger filling space of the bone in the main body 1 is defined.

Also, a center portion in an inner bottom surface of the pressing body 2 has concave conical shape, so that it guides a front end of fastening body 4 precisely to the center of the pressing body 2, and therefore the center of pressing body 2 could receive all the pressing force from the fastening body 4.

The closing body 3 serves primarily to block the rear end of main body 1, since a threaded part 31 formed on the outer circumferential surface of the closing body 3 is engaged with the threaded part 14 of the rear part in the main body 1.

This closing body 3 substantially becomes an auxiliary member for the coupling of the fastening body 4, and therefore is provided with a threaded part 32 for coupling which is formed on an inner circumferential surface thereof so as to serve as a seating surface for the operation driver as well as a passage for filling the bone. The threaded part 32 is closed by the fastening body 4, after the filling of the bone into the main body 1 through the threaded part 32.

The fastening body 4 serves to push the pressing body 2 using the force generated when the fastening body 4 is coupled to closing body 3.

A threaded part 41 to be coupled into the threaded part 32 of the closing body 3 is formed on an outer circumferential surface of a rear portion in the fastening body 4, so that the threaded part 4 pushes the pressing part 2 by a distance proceeding while the threaded part 41 is coupled into the threaded part 32.

In addition, a seating surface 42 for the operation driver is provided on an inner circumferential surface the rear portion in the fastening body 4, and this seating surface 42 could be the same to that of the closing body 3.

Further, in the fastening body 4, an upper part is formed as a lengthy pressing rod 43 of a small diameter, which occupies almost the fastening body 4, and a spiral blade 44 is formed continuously on an outer circumferential surface of the pressing rod 43 with a constant pitch. This spiral blade 44 serves to push a bony materials or fusion facilitating agents filled around toward an outer side, i.e. a wall of the main body 1 while rotating according to fastening body 4 rotation for coupling, so that a filling density of bone in the main body 1 is increased and the fusion of the bony materials and the bodies of spine is facilitated effectively through the holes 16.

The procedure inserting the spine fusion implant BI according to the present invention between the bodies of spine is as follows.

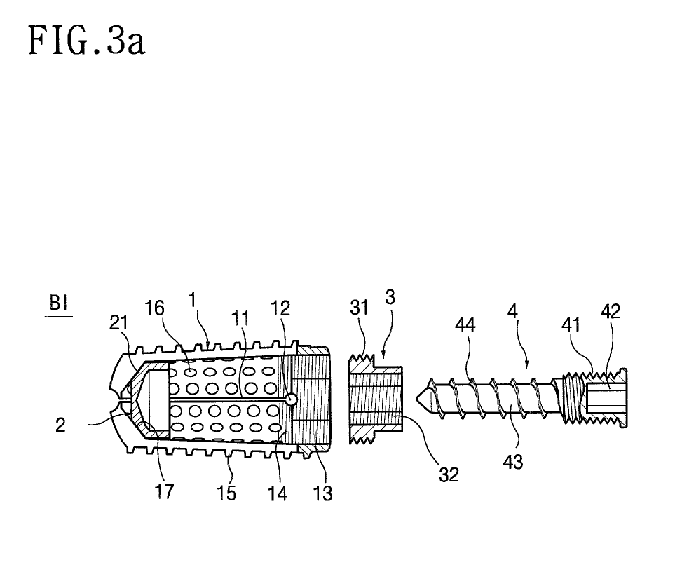

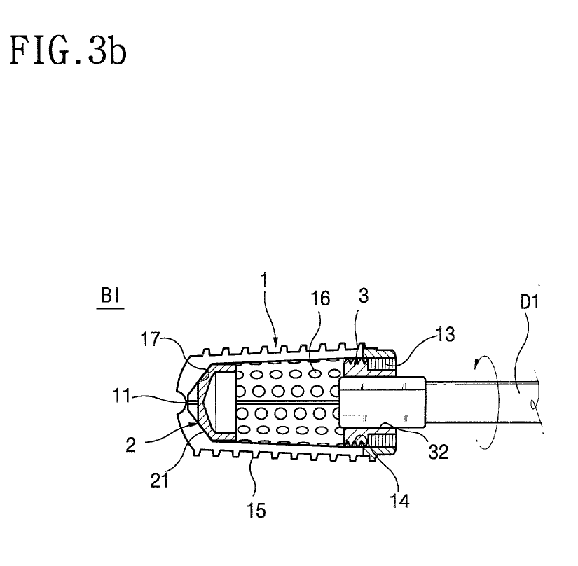

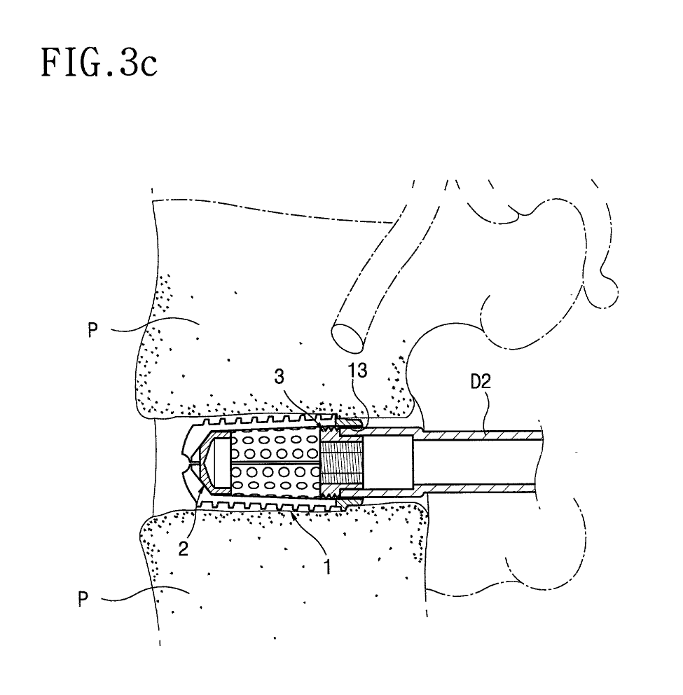

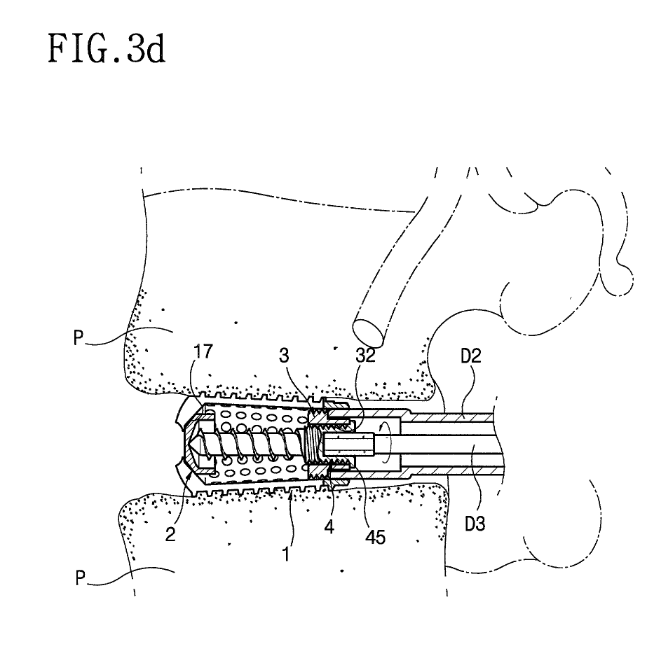

As shown in FIG. 3a, to insert the spine fusion implant BI according to the present invention between the bodies P and P, the pressing body 2 is coupled temporarily on the compressed surface 17 of the main body 1, and the closing body 3 and fastening body 4 are separated from the main body 1.

In such a state, as shown in FIG. 3b, the inner space of main body 1 is filled with crushed bones taken from an operated part of the patient, and the closing body 3 is coupled to the threaded part 14 of main body 1 using the operation driver D1. This procedure occurs out of the corresponding operated part of patient so that it is conducted without any difficulty and the front end of the main body 1 is not deformed.

And, then as shown in FIG. 3c, the main body 1, pressing body 2 and closing body 3 are rotated by an operation driver D2 which is coupled on the driver mounting surface 13 of the main body 1, and then are inserted into a bore formed in a disk space between the adjacent bodies P and P from the rear of spine. The operation driver D2 used for the insertion of the main body 1 is hollow one.

In the insertion procedure described above, the main body 1 could be inserted easily since the front end of a relatively small diameter is inserted firstly.

When the insertion of the main body 1 is completed, as shown in FIG. 3d, the fastening body 4 is inserted in the main body 1 through an inner space of the hollow operation driver D2 and threaded part 32 of the closing body 3, and then the fastening body 4 is coupled to the threaded part 32 of the closing body 3 using an operation driver D3 inserted into the operation driver D3.

During the coupling of the fastening body 4, the front end thereof contacts with the conical center portion of the pressing body 2 from an initial step of coupling, and the pressing body 2 slides on the compressed surface of main body 1 and is pushed toward the front end of the main body 1 in proportion to the coupling extent of the fastening body 4.

When the pressing body 2 is pushed toward the front end of the main body 1, the front end of the main body 1 is expanded. Furthermore, according to the rotation of fastening body 4 the spiral blade 44 pushes and disperses the bone filled in the main body 1 to the outside though the holes 16 for fusion so that the bone in the main body 1 contacts with the bodies and the bone fusion after the operation is facilitated stably.

When the fastening body 4 is coupled completely, i.e. a rear flange of the fastening body 4 is engaged with the rear end of the closing body 3, the front end of the main body 1 is expanded suitably in a relatively wide front space between an upper and lower bodies P and P. That is, the spine fusion implant BI is inserted suitable for both wide front space and relatively narrow rear space and is seated so as to support the upper and lower bodies P and P. In such a state, the insertion procedure of the spine fusion implant is completed.

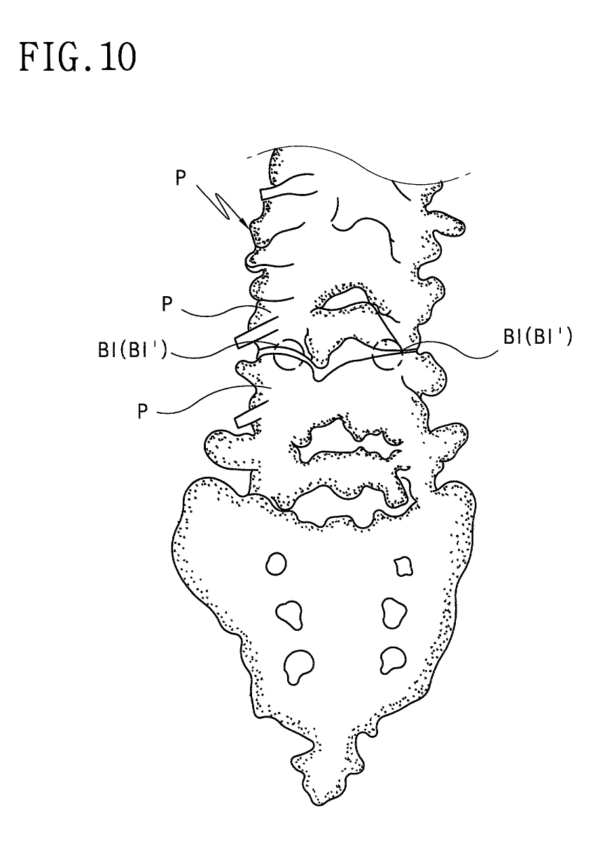

The spine fusion implants BI according to the present invention, as shown in FIG. 10, are inserted into both spaces between the bodied P and P so as to support the upper and lower bodies P and P to be stable and balanced.

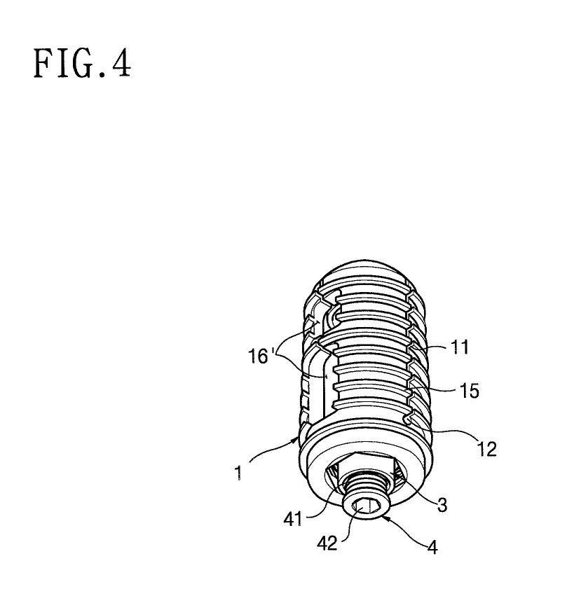

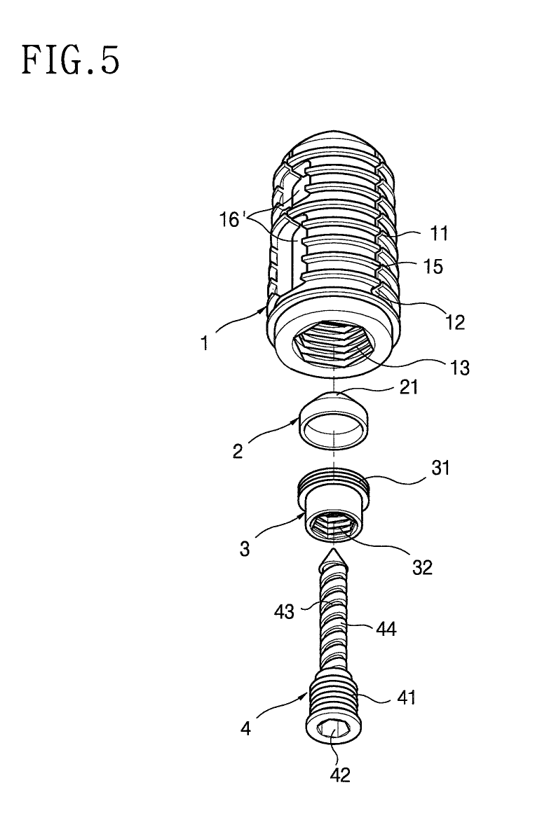

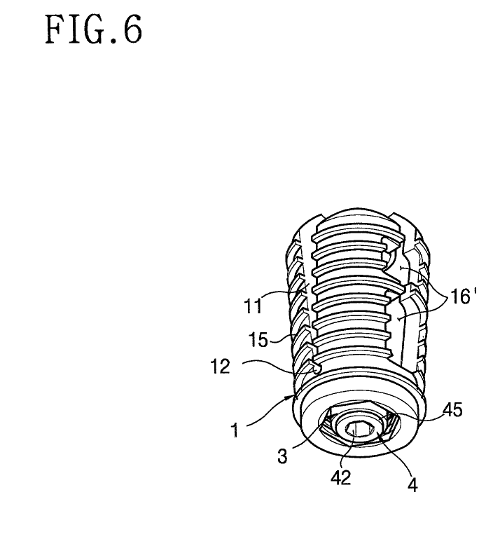

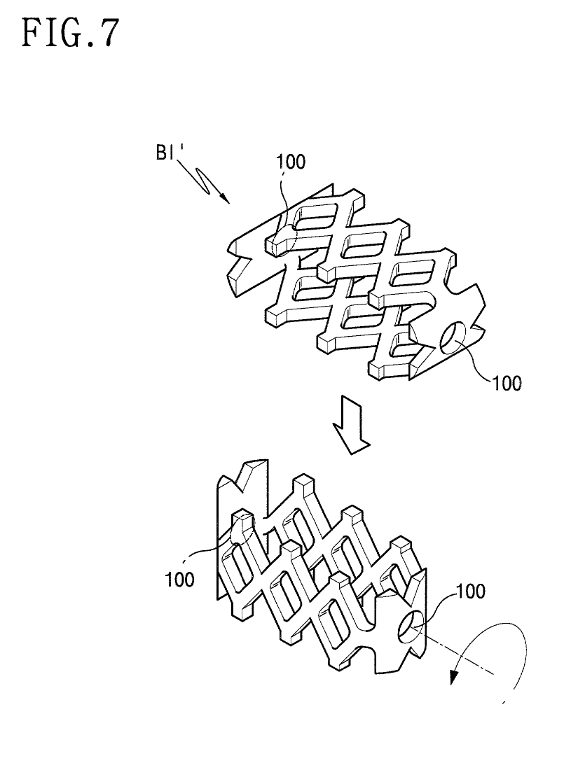

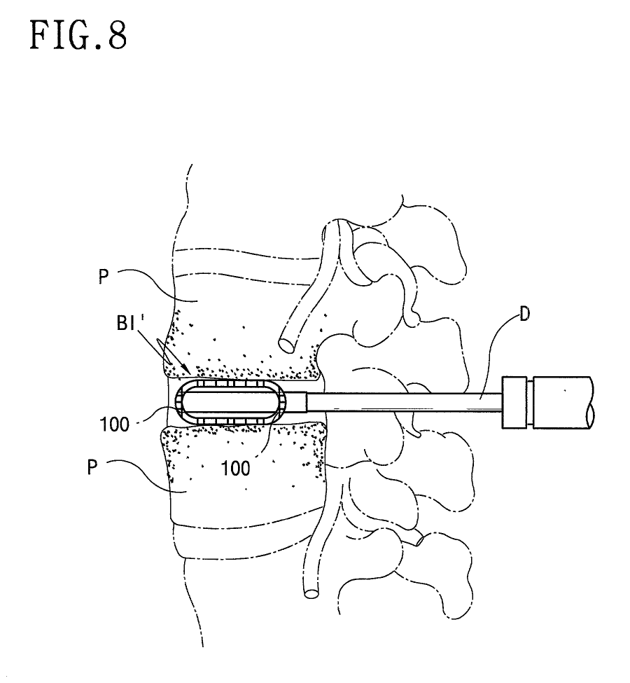

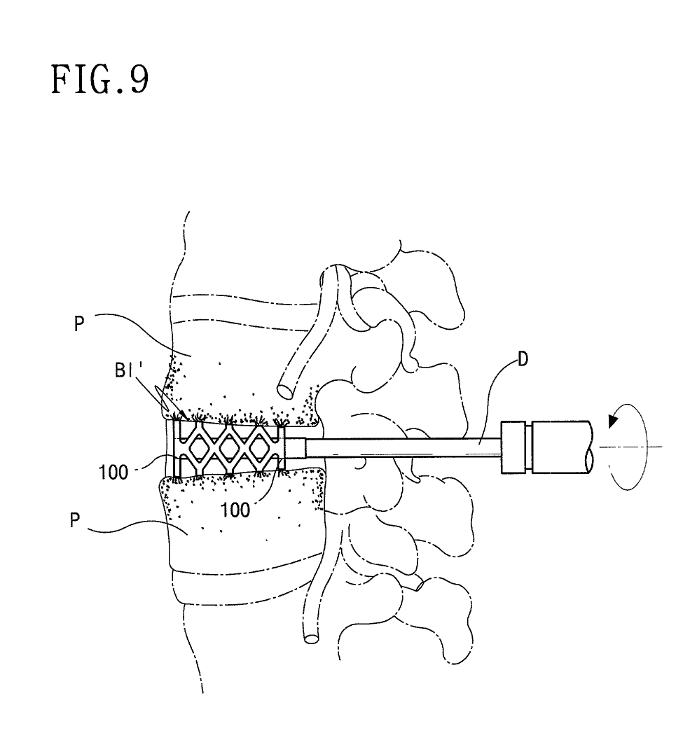

As shown in FIG. 4-FIG. 6, the main body 1 could be provided with at least one pair of rectangular holes 16′ for bone fusion which is arranged symmetrically, as an another embodiment, and it is apparent that this falls into the scope of the present invention.

The advantages of the present invention are summarized as follows.

The spine fusion implant according to the present invention is seated suitable for the front and rear space between the adjacent spine bodies by expanding the front end thereof after the completion of insertion, define the maximum inner space and simultaneously enables the bone filled therein to contact directly with the spine bodies so as to prepare a advantageous condition for bone fusion. Therefore, the improper damage of spine bodies perfectly, and the operation is performed with the implant body filled with sufficient amount of the bone before the insertion.

Further, the spine fusion implant of the present invention defines the inner space filled with the bone widely as possible, and disperse the bone filled therein toward the peripheral spine bodies and increases the filling density of the bone. Accordingly, the spine fusion implant and the spine bodies are fused stably and rapidly, the bone growth and spine stability is increased, and a doctor could retain composure due to the easiness of the operation so that the entire spine fusion operation is performed safely and effectively.

Although a number of embodiment have described in the above specification, it should be apparent that the present invention could be embodied in many other specific mode included within the sprit and scope of the present invention. Thus, the present embodiments should be considered as illustrative, and the present invention could be modified within the scope of claims and the equivalent thereof.

What is claimed is:

1. A spine fusion implant comprising:
a cylindrical main body having an extendable front end;
a pressing body of a cap shape expanding the front end of the main body by receiving a pulling force;
a closing body coupled to a rear end of the main body and serving as a guide member for a coupling of a fastening body;
the fastening body coupled to the closing body and pulling the pressing body.

2. The spine fusion implant according to claim 1, wherein means for enabling an expansion of the front end in the main body is an incision part which is incised at least one direction.

3. The spine fusion implant according to claim 1, wherein a driver mounting surface is formed on an inner circumferential surface of the rear end in the main body and a threaded part is formed at an inner side of the driver mounting surface.

4. The spine fusion implant according to claim 1, wherein a spiral rib is formed continuously along a length direction on a surface of the main body.

5. The spine fusion implant according to claim 1, wherein a plurality of holes for bone fusion are formed on entire surface of the main body.

6. The spine fusion implant according to claim 1, wherein a diameter of the front end in main body is smaller than that of the rear end.

7. The spine fusion implant according to claim 1, wherein the pressing body comprises a cap member having an inclined pressing surface at a front end thereof and the pressing surface is fixed temporarily by inserted on a compressed surface formed at an inner surface of the front end in the main body.

8. The spine fusion implant according to claim 1, wherein a threaded part for coupling with the fastening body and filling the bone into the main body is formed to penetrates a center portion of the closing body, and a threaded part for coupling with the main body is formed on an outer circumferential surface of the closing body.

9. The spine fusion implant according to claim 1, wherein a threaded part for coupling with the closing body is formed on an outer circumferential surface of a rear portion in the fastening body, a driver seating surface is formed on an inner circumferential surface of the rear portion in the fastening body, and a lengthy cylindrical pressing rod is formed to be extended from the threaded part of the fastening body

10. The spine fusion implant according to claim 1, wherein a continuous spiral blade is provided on an outer circumferential surface of the pressing rod.
